(19) United States
(12) Patent Application Publication
(10) Pub. No.: US 2023/0267612 A1
(43) Pub. Date: Aug. 24, 2023

(54) A monitoring system

(71) Applicant: Sanofi, Paris (FR)

(72) Inventors: Michael Helmer, Frankfurt am Main (DE); Martin Vitt, Frankfurt am Main (DE)

(21) Appl. No.: 18/018,345

(22) PCT Filed: Aug. 11, 2021
(86) PCT No.: PCT/EP2021/072332

§ 371 Date: Jan. 27, 2023

(30) Foreign Application Priority Data
Aug. 14, 2020 (EP) 20315385.3

(51) Int. Cl. G06T 7/00; G06V 40/16; G06V 10/75; G06T 7/90; G06T 7/62

(52) U.S. Cl. CPC G06T 7/0016; G06V 40/171; G06V 10/751; G06T 7/90; G06T 7/62; G06T 2207/30201; G06T 2207/30041; G06T 2207/30088; G06V 2201/03

(57) ABSTRACT

An apparatus for indicating health conditions of a user is disclosed. The apparatus includes a camera configured to capture a reference image of a user's face and a new image of a user's face. The apparatus also includes a processor configured to determine facial properties of the user in the reference image and the new image; determine any differences in the facial properties determined from the reference image and the status image; generate a warning when differences in the facial properties between the reference image and the new image are determined; and store the record in a memory. A method of facial recognition for indicating health conditions of a user and a computer program comprising machine readable instructions are also provided.

### CROSS REFERENCE TO RELATED APPLICATIONS

The present application is the national stage entry of International Patent Application No. PCT/EP2021/072332, filed on Aug. 11, 2021, and claims priority to Application No. EP 20315385.3, filed on Aug. 14, 2020, the disclosures of which are incorporated herein by reference.

### TECHNICAL FIELD

The present disclosure relates to a monitoring system for patients during drug treatment and in particular, a facial monitoring system.

### BACKGROUND

A variety of diseases exist which require regular treatment, for instance, by injection of a medicament. Such injections can be performed by using injection devices, which are applied either by medical personnel or by patients themselves. As an example, type-1 and type-2 diabetes can be treated by patients themselves by injection of insulin doses, for example once or several times per day. Similarly, at a preliminary stage before diabetes, patients who are overweight or obese can administer by injection treatment for chronic weight management.

Unfortunately, in the course of drug treatment the patient may experience side-effects, which may result either from the drugs themselves or the condition for which they are being treated. These side-effects could include, for instance, weight gain, greater susceptibility to contracting other diseases or an overall deterioration in general health conditions. In some instances, these side-effects may present themselves as changes in the properties or characteristics of the patient's face.

### SUMMARY

Where patients require regular or long-term treatment, there is a need to provide a system that can monitor the patient's overall well-being in response to that treatment. In particular, there is a need to determine any changes in the patient's health in response to a treatment regimen and to encourage good patient habits in adhering to the treatment regimen. This is important as changes in the patient's response to the treatment may require, for instance, the nature of the treatment, such as the dosing regimen, to be adjusted or additional treatment to be introduced.

Aspects of the present disclosure have been conceived with the foregoing in mind.

According to an aspect of the present disclosure, there is provided a computer program comprising machine readable instructions that when executed by a processor, causes the processor to control a camera to capture a reference image of a user's face and a new image of a user's face; determine facial properties of the user in the reference image and the new image; determine any differences in the facial properties determined from the reference image and the new image; generate a record of the facial properties; generate a warning when differences in the facial properties between the reference image and the new image are determined; and store the record in a memory.

This is advantageous as it provides a means by which to monitor and track a patient's overall well-being in response to a treatment. In addition, it is possible to identify any changes in the patient's condition in response to treatment and/or the development of secondary diseases. The computer program thereby provides an early warning system for changes in patient health and compliance with the treatment regimen.

The facial properties may relate to at least one of eyes, skin, hair, and facial impression.

The processor may also determine a facial impression based on the distance measured between a fixed face point and a variable face point.

The fixed face point may comprise at least one of the bridge of the nose and an outer edge of a nostril. The variable face point may comprise at least one of an outer edge of an eyelid and a corner of the mouth.

The processor may determine at least one of the colour and clarity of eye dermis.

The processor may determine at least one of skin colour, skin tone and skin moisture.

The processor may determine at least one of hair distribution and hair volume.

The processor may control a display to display the warning, and the warning includes a user survey.

The processor may control a communication unit to transmit the survey to an external device when the survey is completed.

The processor may, prior to capturing the reference image and/or the new image, generate an input window requesting the reference image or the new image is taken, and control a display to display the input window.

According to another aspect of the present disclosure, there is provided a smart phone application comprising a computer program according to the present disclosure.

According to another aspect of the present disclosure, there is provided an apparatus for indicating health conditions of a user comprising a camera configured to capture a reference image of a user's face and a new image of a user's face; and a processor configured to determine facial properties of the user in the reference image and the new image; determine any differences in the facial properties determined from the reference image and the status image; generate a warning when differences in the facial properties between the reference image and the new image are determined; and store the record in a memory.

The apparatus may be a mobile device.

According to another aspect of the present disclosure, there is provided a method of facial recognition for indicating health conditions of a user comprising capturing a reference image of a user's face and a new image of a user's face; determining facial properties in the reference image and in the new image; determining differences in the facial properties determined from the reference image and the new image; generating a record of the facial properties; generating a warning when differences in the facial properties between the reference image and the new image are determined; and storing the record in a memory.

Embodiments of the disclosure will now be described, by way of example only, with reference to the accompanying drawings.

### BRIEF DESCRIPTION OF THE FIGURES

In the Figures:

FIG. 1 is a schematic showing internal components of a mobile device.

FIG. 2 is a flow chart showing operation of the monitoring system during an initial set-up process.

FIG. 3 is a flow chart showing operation of the monitoring system during subsequent use, for instance, on a daily basis.

FIG. 4 shows the fixed face points and variable face points identified by the evaluation tool employed during facial analysis.

FIG. 5 shows the skin target areas identified by the evaluation tool employed during facial analysis.

### DETAILED DESCRIPTION

In the following disclosure a computer program will be described having machine readable instructions that when executed by a processor causes the processor to initiate various operations.

In the following exemplary embodiment the computer program is implemented in a mobile device and the computer program is in the form of a monitoring application.

In brief, the computer program as implemented in the mobile device provides a monitoring system or monitoring function that monitors change in a patient's facial impression during drug treatment. When executed, the monitoring application provides, for instance, indications of the overall well-being of the patient in response to drug treatment. The monitoring application could be provided to support a variety of clinical studies. These could include, for instance, clinical studies related to diabetes, chronic weight control, cardio vascular conditions, rheumatism or psoriasis.

According to the following exemplary embodiment, the mobile device is a mobile phone, such as a smartphone. Advantageously, the monitoring application is a distinct application. The monitoring application may be provided in the mobile device on manufacture or may be downloaded into the mobile device by a user, for instance from an application market place or application store.

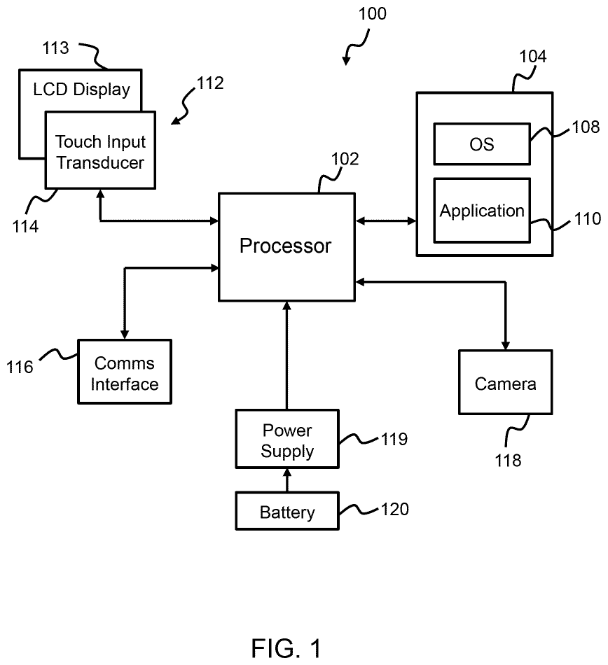

FIG. 1 is a schematic of some of the internal components of the mobile device 100. The mobile device 100 includes a processor 102. The processor 102 may be an integrated circuit of any kind. The processor 102 controls operation of the other hardware components of the mobile device 100. The processor 102 and other hardware components may be connected via a system bus (not shown). Each hardware component may be connected to the system bus either directly or via an interface.

The mobile device 100 comprises a display 112 (for instance an LCD, TFT (thin film transistor), OLED (organic light emitting diode), ePaper). The display may be a touch sensitive display having a display part 113 and a tactile interface part 114. The mobile device 100 also includes a communications interface 116, such as a Bluetooth interface. The mobile device 100 also comprises a camera 118. Any suitable camera may be employed and the camera 118 may include a front facing lens and/or a rear facing lens. The mobile device 100 may also comprise a radar sensor (not shown). The radar sensor may be able to perform facial recognition, for instance, by detecting and recording changes in facial features, such as facial contours or dimensions. The radar sensor may thereby contribute to the operation of the camera 118 in performing facial recognition. The mobile device 100 also houses a battery 120 to power the mobile device 100 by a power supply 119.

The processor 102 is configured to send and receive signals to and from the other components in order to control operation of the other components. For example, the processor 102 controls the display of content on the display 112 and receives signals as a result of user inputs from the tactile interface 114. The display 112 may be a resistive touch screen or capacitive touch screen of any kind. The display 112 may alternatively not be a touch screen. For instance, the display 112 may be a liquid crystal display (LCD).

The mobile device 100 comprises a memory 104, i.e. a working or volatile memory, such as Random Access Memory (RAM), and a non-volatile memory. The processor 102 may access RAM in order to process data and may control the storage of data in memory 104. The RAM may be a RAM of any type, for example Static RAM (SRAM), Dynamic RAM (DRAM) or a Flash memory. The non-volatile memory stores an operating system 108 and the monitoring application 110, as well as storing data files and associated metadata. The memory 104 may be a non-volatile memory of any kind such as a Read Only Memory (ROM), a flash memory and a magnetic drive memory.

The processor 102 operates under control of the operating system 108. The operating system 108 may comprise code relating to hardware such as the display 112 and the communications interface 116, as well as the basic operation of the mobile device 100. The operating system 108 may also cause activation of other software modules stored in the memory 104, in addition to or instead of the monitoring application 110.

Other standard or optional components of the mobile device 100, such as transceivers, are omitted.

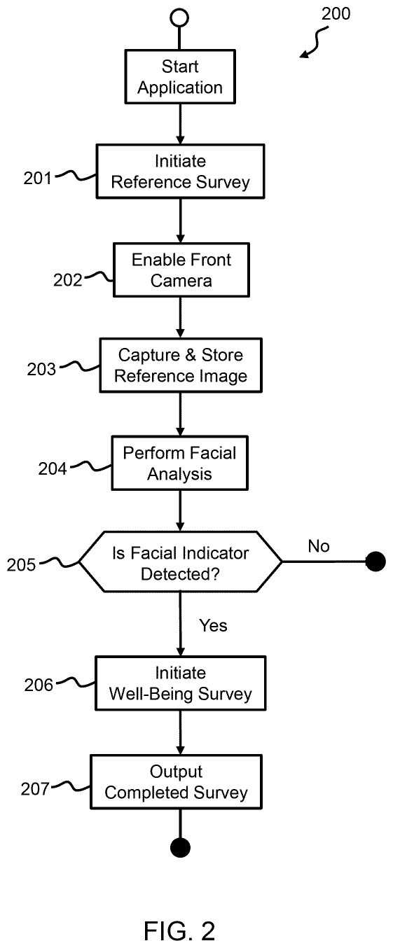

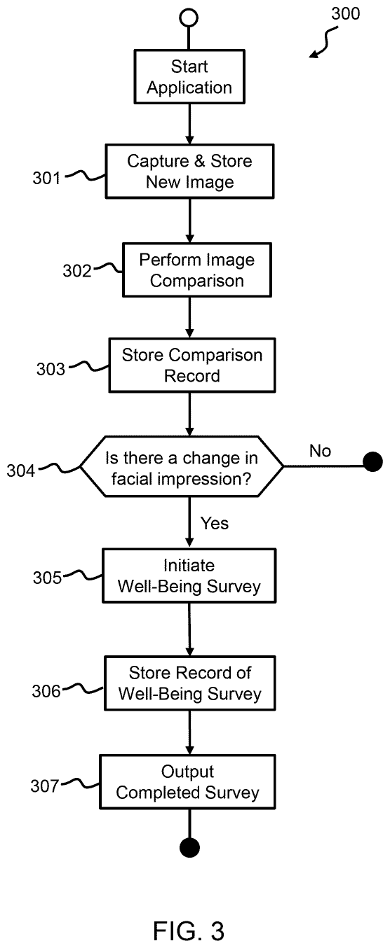

FIGS. 2 and 3 are flow charts illustrating operation of the monitoring application 110 when the machine readable instructions are executed by the processor 102 of the mobile device 100. The flow charts indicate how the monitoring application 110 and the mobile device 100 interact and operate to provide a monitoring system. The steps are performed by the processor 102 of the mobile device 100 under control of the monitoring application 110 stored in the memory 104.

FIG. 2 details the initial operation or setting-up process of the monitoring application 110. In FIG. 2, the operation 200 starts when the monitoring application 110 is initiated for the first time. In step 201, an initial reference survey is initiated to gather information regarding the general health conditions of a patient.

The survey may include questions that assess the physical function, pain or overall health of the patient, for instance. The survey may include questions associated with, or relevant to, one or more particular clinical studies. For example, rheumatism patients could be requested to answer questions according to the RAPID 3 checklist. As another example, questions for diabetes patients may focus on monitoring typical side effects in order to improve drug titration or to find the correct dose and time for injection. As a further example, questions may focus on monitoring secondary diseases that commonly result from a patient's primary disease. For instance, cardio diseases associated with diabetes or psoriasis arthritis associated with rheumatoid arthritis. However, the above examples are not intended to be limiting and any suitable questions relevant to general patient health conditions could be included.

In step 202, the camera 118 of the mobile device 100 is enabled. For instance, a request is displayed asking for permission for the monitoring application 110 to access and enable the camera 118 so that at least one image of the user can be captured by the camera 118. The camera 118 may be front facing, for example, so that an image is taken whilst the user is viewing the display 112 of the mobile device 100. In step 203, the camera 118 captures a reference image of the user's face and head and stores the reference image. The reference image provides a starting point for comparison against any future images captured by the camera 118 (FIG. 3). The reference image is stored, for instance, in the memory 104 of the mobile device 100.

In step 204, facial analysis of the reference image is performed. This may involve, for instance, determining the characteristics (properties) of the user's face. The facial properties determined may then be compared against pre-stored information detailing pre-defined facial characteristics that are indicative of diseases, disorders or drug side-effects. This comparison may identify one or more facial indicators. A facial indicator represents a facial characteristic that is indicative of a disease, disorder, drug side-effect or other general change in the user's health condition.

If a facial indicator is not identified then, in step 205, no further action is required and the monitoring application 110 may be closed.

If a facial indicator is identified in step 205, then a well-being survey (user survey) is initiated in step 206. In other words, a warning is generated in the form of the well-being survey. The well-being survey may be regarded as a secondary survey to the reference survey which focuses on the change in the user's facial characteristics. The well-being survey may request further details regarding the health of the patient. The well-being survey may, for instance, request further information relevant to the condition indicated by the one or more facial indicators identified in the facial analysis. In other words, the well-being survey is configured to try and discover further information relating to the patient's health that could be linked to the change in condition indicated by the facial analysis.

In step 207, the completed well-being survey is stored. The completed survey may be stored at the mobile device 100, for instance, in the memory 104 of the mobile device 100. In step 207, the completed survey is transmitted (output) to an external device. The external device may, for instance, include a server. The server could be a server that can be accessed by health professionals. Once the survey has been transmitted no further action is required and the monitoring application 110 may be closed.

FIG. 3 details the daily operation of the monitoring application 110, once the initial set-up is complete (FIG. 2). In FIG. 3, the operation 300 starts when the monitoring application 110 is initiated. This may be at any time according to the user. In step 301, the monitoring application 110 captures and stores an image of the user's face and head. The image captured represents a new image in relation to the reference image captured during the initial operation of the monitoring application 110 (FIG. 2, step 203).

In step 302, image comparison is performed in which the new image is compared with the reference image to determine if there are any changes in the facial characteristics of the user. In this comparison, facial analysis of the new image is performed to determine if there are any differences in the facial properties determined from the new image compared with those determined from the reference image. A record of the facial properties determined from the new image is generated and the results of the record are compared against the results of facial analysis carried out with respect to the reference image (FIG. 2, step 204). This may involve, for instance, analysing the characteristics of the user's face against pre-stored information detailing pre-defined facial characteristics that represent facial indicators which could be indicative of diseases, disorders or drug side-effects.

In step 303, the results of the comparison are stored (comparison record). The results may be stored in the form of a health record. The record may be stored at the mobile device 100, for instance, in the memory 104 of the mobile device 100.

If no changes are detected in the facial characteristics of the user in the new image, such that one or more facial indicators indicative of a disease is not identified in the characteristics of the user's face then, in step 304, no further action is required and the monitoring application 110 may be closed.

If a change in one or more characteristics of the user's face is detected, in step 304, such that one or more facial indicators indicative of a disease, disorder, drug side-effects or other general change in the user's health condition is identified, then a well-being survey (user survey) is initiated in step 305. In other words, when difference in the facial properties between the reference image and the new image are determined a warning is generated in the form of the well-being survey. The well-being survey may request further details regarding the health of the patient. The well-being survey may, for instance, request further information relevant to the condition indicated by the one or more facial indicators identified in the facial analysis. In other words, the well-being survey is configured to try and discover further information relating to the patient that could be linked to the condition indicated by the facial analysis.

In step 306, the completed well-being survey is stored. The completed survey may be stored at the mobile device 100, for instance, in the memory 104 of the mobile device 100. In step 307, the completed survey is transmitted (output) to an external device. The external device may, for instance, include a server. The server may be accessible by a health professional. Once the survey has been transmitted no further action is required and the monitoring application 110 may be closed.

The facial analysis performed by the monitoring application 110 in step 204 of FIG. 2 and step 302 of FIG. 3 will now be described in more detail. The facial analysis may be any suitable form of facial analysis for determining characteristics or properties of a user's face. For instance, the facial analysis may include face recognition implemented by software algorithms that evaluate image data. Radar sensor data may also be evaluated in combination with the image data.

The characteristics or properties of the user's face may include, for instance, eyes, skin, hair, locations of features and the overall facial impression of the user. A facial indicator is a facial characteristic that is indicative of diseases, disorders or drug side-effects that are, for instance, in either a preliminary stage of development or a well-established stage of development.

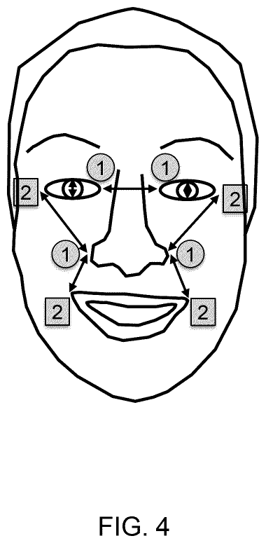

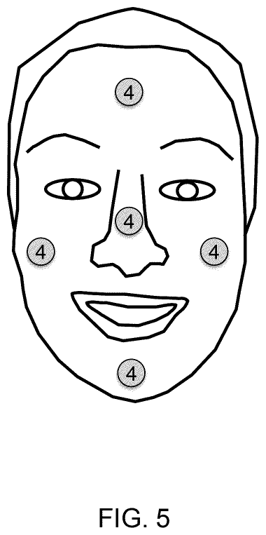

FIGS. 4 and 5 show images of target areas of a user's face used during facial analysis.

In FIG. 4, exemplary fixed face points 1 and variable face points 2 are identified on a user's face. Fixed face points 1 represent locations on a user's face that are unlikely to change. These locations may include, for instance, the inner points of the eye near the nose where tear ducts are located, the bridge of the nose or an outer edge of a nostril. In contrast, variable face points 2 represent locations on a user's face that may change, for instance, in response to the expression of the user (e.g. an emotional expression such as smiling when happy or frowning when unhappy). These locations may include, for instance, an outer edge of an eye or eyelid or a corner of the mouth.

In facial analysis, an evaluation tool is configured to measure distances between fixed face points 1 and variable face points 2. The distances measured between fixed face points 1 and variable face points 2 may provide an indication as to the overall facial impression of the user. The evaluation tool measures a variety of distances, for instance, the distance between the eyes across the bridge of the nose, the height of the eye from the top eyelid to the bottom eyelid, the distance from the outer nostril to the outer edge of the eye, or the distance between the outer nostril and the outer side edge of the lip. Facial indicators associated with the fixed and variable face points 1, 2 may include a drooping mouth, cheeks or eyes.

The evaluation tool may also measure eye characteristics. Eye characteristics may include, for instance, the colour and/or clarity of the eye dermis or the colour and/or form of the eyelids. Facial indicators associated with the eyes may include, for instance, dry, red, and/or discoloured eyes or red and/or swollen eyelids.

The evaluation tool may also measure skin characteristics. Target skin areas are shown in FIG. 5. The evaluation tool detects changes in the skin in the target areas. Skin characteristics may include, for instance, colour, tone or presence of moisture (e.g. sweating). Facial indicators associated with the skin may include, for instance, redness or excess moisture.

The evaluation tool also measures hair characteristics. The evaluation tool may detect, for instance, changes in the distribution and volume of the hair. Facial indicators associated with hair may include, for instance, hair loss, a receding hair line, or development of excess facial hair.

The facial characteristics identified during facial analysis are compared against pre-stored information detailing pre-defined facial characteristics. Those characteristics which could be indicative of diseases, disorders or drug side-effects are then classified as facial indicators.

In addition, in the course of drug treatment a number of images and well-being surveys may be taken and stored. When the number of stored images and/or surveys is equal to or greater than a pre-determined value then the monitoring system may generate a new reference image or determine new facial characteristics to be assessed during facial analysis. For instance, the new reference image may represent an ideal reference image that represents a user's actual (base line) appearance without any emotional influences, such as being happy, tired or angry. The new reference image can thereby provide a more effective diagnostic evaluation.

Various alterations and modifications to the embodiments described above will now be discussed.

The present disclosure is described with reference to diabetes and chronic weight management, but this is not intended to be limiting and the teaching herein may equally well be deployed with respect to other diseases or health conditions.

The present disclosure is described in the context of a computer program implemented in a mobile device 100, but this is not intended to be limiting and the computer program may equally well be implemented in another suitable apparatus. For instance, the apparatus may equally well be implemented in another mobile device 100, such as a PDA, a tablet computer of any kind, or a medical device, such as a blood glucose meter device. Alternatively, the computer program may be implemented in another suitable apparatus, such as a PC.

The well-being survey is described as being initiated in step 206, but the initial well-being survey may equally well be initiated after the camera 118 is enabled in step 202 (FIG. 2). In addition, the reference image (step 203) and/or the completed well-being and/or discovery survey (step 206, 306) may be transmitted and stored at an external device (FIGS. 2 and 3). Optionally, an input window requesting that the reference image and/or the new image is taken is displayed prior to capturing the reference image and/or new image in steps 203 and 301, respectively.

The evaluation tool may measure any combination of one or more of the facial characteristics described.

The facial indicators described are exemplary and do not represent an exhaustive list and other characteristics may also represent facial indicators.

1-15. (canceled)

16. A computer program comprising machine readable instructions that when executed by a processor, causes the processor to:
control a camera to capture a reference image of a user's face and a subsequent image of the user's face;
determine reference facial properties of the user from the reference image, and subsequent facial properties of the user from the subsequent image;
determine any differences between the reference facial properties and the subsequent facial properties;
generate a record of the reference and the subsequent facial properties;
generate a warning when the differences between the reference facial properties and the subsequent facial properties are determined; and
store the record in a memory.

17. The computer program according to claim 16, wherein each of the reference facial properties and the subsequent facial properties relate to at least one of eyes, skin, hair, or facial impression of the user as depicted in a respective one of the reference image and the subsequent image.

18. The computer program according to claim 17, wherein the instructions cause the processor to determine a respective facial impression from each of the reference image and the subsequent image based on a respective distance measured between a fixed face point and a variable face point depicted in the corresponding image.

19. The computer program according to claim 18, wherein the fixed face point comprises at least one of a bridge of a nose and an outer edge of a nostril.

20. The computer program according to claim 18, wherein the variable face point comprises at least one of an outer edge of an eyelid and a corner of a mouth.

21. The computer program according to claim 17, wherein the instructions cause the processor to determine at least one of a respective color or clarity of eye dermis from each of the reference image and the subsequent image.

22. The computer program according to claim 17, wherein the instructions cause the processor to determine at least one of a respective skin color, skin tone, or skin moisture from each of the reference image and the subsequent image.

23. The computer program according to claim 16, wherein the instructions cause the processor to determine at least one of a respective hair distribution, or hair volume from each of the reference and the subsequent images.

24. The computer program according to claim 16, wherein the instructions cause the processor to control a display to display the warning, and the warning includes a user survey.

25. The computer program according to claim 24, the instructions cause the processor to control a communication unit to transmit the user survey to an external device when the user survey is completed.

26. The computer program according to claim 16, wherein prior to capturing the reference image and/or the subsequent image, the instructions cause the processor to:
generate an input window requesting the reference image or the subsequent image to be taken, and
control a display to display the input window.

27. An apparatus for indicating health conditions of a user comprising:
a camera configured to capture a reference image of a face of the user and a subsequent image of the face of the user; and
a processor configured to:
determine reference facial properties of the user from the reference image, and subsequent facial properties of the user from the subsequent image,
determine any differences between the reference facial properties and the subsequent facial properties,
generate a record of the reference and the subsequent facial properties,
generate a warning when the differences between the reference facial properties and the subsequent facial properties are determined, and
store the record in a memory.

28. The apparatus according to claim 27, wherein the apparatus is a mobile device.

29. The apparatus according to claim 27, wherein each of the reference facial properties and the subsequent facial properties relate to at least one of eyes, skin, hair, or facial impression of the user depicted in a respective one of the reference image and the subsequent image.

30. The apparatus according to claim 29, wherein the processor is configured to determine a respective facial impression from each of the reference image and the subsequent image based on a respective distance measured between a fixed face point and a variable face point depicted in the corresponding image.

31. The apparatus according to claim 30, wherein the fixed face point comprises at least one of a bridge of a nose and an outer edge of a nostril, and
wherein the variable face point comprises at least one of an outer edge of an eyelid and a corner of a mouth.

32. The apparatus according to claim 27, wherein the processor is configured to determine at least one of a respective skin color, skin tone, skin moisture, hair distribution, hair volume, eye color and/or clarity of eye dermis from each of the reference image and the subsequent image.

33. The apparatus according to claim 27, further comprising a user interface configured to display the warning, and the warning including a user survey.

34. The apparatus according to claim 33, wherein the user interface is further configured to receive user inputs for the user survey, and
wherein the apparatus further comprises a transceiver configured to transmit the user survey to an external device when the user survey is completed.

35. A method of facial recognition for indicating health conditions of a user comprising:
capturing a reference image of a face of the user and a subsequent image of the face of the user;
determining reference facial properties of the user from the reference image, and subsequent facial properties of the user from the subsequent image;
determining differences between the reference the facial properties and the subsequent facial properties;
generating a record of the reference facial properties and the subsequent facial properties;
generating a warning when the differences between the reference facial properties and the subsequent facial properties are determined; and
storing the record in a memory.
